SEQUENCE LISTING (2 sequences)

SEQ ID NO: 1 - DNA, 18 bp, Artificial sequence
  Randomly chosen: There is no significance to sequence
uuuuactgcgtgtgcagg

SEQ ID NO: 2 - DNA, 18 bp, Artificial sequence
  Randomly chosen: There is no significance to sequence
uuuugtcgatgcgtgcgt

(19) United States
(12) Patent Application Publication
(10) Pub. No.: US 2009/0142759 A1
(43) Pub. Date: Jun. 4, 2009

(54) qPCR array with IN SITU primer synthesis

(76) Inventor: Erik Larsson, Gothenburg (SE)

Correspondence Address: LYNN E BARBER, P O BOX 16528, FORT WORTH, TX, 76162, US

(21) Appl. No.: 11/998,267

(22) Filed: Nov. 29, 2007

(51) Int. Cl. C12Q 1/68

(52) U.S. Cl. 435/6

(57) ABSTRACT

Application of in situ oligonucleotide synthesis, using a maskless photolithographic oligonucleotide synthesis apparatus or by other means, for direct fabrication of polymerase chain reaction (PCR) primers in situ in PCR reaction wells. The synthesized oligonucleotides contain an enzymatically degradable linker sequence and a specific primer sequence. The method may be used for manufacturing of quantitative PCR (qPCR) arrays containing a plurality of independent qPCR assays while eliminating the need for presynthesized primer libraries.

### FIELD OF THE INVENTION

The present invention relates generally to the application of in situ oligonucleotide synthesis, for direct fabrication of polymerase chain reaction (PCR) primers in situ in PCR reaction wells.

### BACKGROUND OF THE INVENTION

DNA microarrays have become standard tools in biological and biomedical research. They enable simultaneous quantification of thousands of specific DNA sequences in parallel. In principle, a microarray consists of small features (probes) of DNA spotted or by other means attached to a flat substrate. Each probe has a unique sequence which is complementary to a certain target DNA sequence. By hybridizing a fluorescently labelled DNA or RNA sample onto the array, the relative amounts of different nucleic acid sequence species in the sample may be determined. Microarrays are used extensively for gene expression profiling in many applications including the discovery of gene function, drug evaluation, pathway dissection and classification of clinical samples. They are also used for highly parallel allele discrimination, e.g. single nucleotide polymorphism genotyping. Arrays may be produced either by deposition of presynthesized DNA material or by in situ oligonucleotide synthesis. The latter approach, where oligonucleotides are assembled directly on the array surface, has several advantages. It eliminates robotized spotting, which is a sensitive step often associated with quality problems. The number of features per surface area can be significantly increased. It also eliminates the need for presynthesis and storage of large DNA libraries.

The polymerase chain reaction (PCR) is widely used for specific detection and quantification of polynucleotides. The method uses a pair of oligonucleotides, “primers”, which specifically bind (anneal) to specific locations on a longer DNA molecule. The region in between and including the primers is copied several thousand fold using a cyclic enzymatic reaction based on the use of a heat-tolerant DNA polymerase. Applications include gene expression analysis, single nucleotide polymorphism (SNP) genotyping and chromatin immunoprecipitation (ChIP) studies. PCR is often used together with either a sequence non-specific fluorescent reporter dye such as SYBR green (Wittwer C T et al., Biotechniques. 1997 January; 22(1):176-81) or a sequence specific fluorescent reporter such as a taqman probe (Heid C A et al., Genome Res. 1996 October; 6(10):986-94). By monitoring the fluorescence in the sample during the reaction, this principally simple extension of PCR provides precise quantitative measurements (quantitative PCR, qPCR, real-time PCR). Real-time PCR is commonly used for validation of findings discovered using DNA microarrays and is considered to be the gold standard for gene expression quantification.

PCR or qPCR is typically performed in plastic 96 or 384 well microtiter plates, each reaction having a volume in the order of 5-50 μl. PCR can however be carried out in very small (nanoliter) volumes. Miniaturization and parallelization of PCR is an area of active research and development. One example is provided in (Dahl A et al, Biomed Microdevices. 2007 June; 9(3):307-14) where the reactions are performed in an array of 1024 distinct 200 nl wells in a polypropylene plate. This type of technology has the potential to combine the sensitivity, precision and wide dynamic range of qPCR with the parallelism of conventional hybridization DNA microarrays. A related system is the BioTrove OpenArray platform (U.S. Pat. No. 6,716,629), where PCR reactions are performed in several thousand nanoliter through-holes in a microscope slide-sized metal plate.

A major problem with the currently proposed array-format systems for qPCR is the application of specific primers (and, where applicable, probes) in each reaction well. For example, in a gene expression application one is often interested in measuring the expression of a large number of different genes in a small number of samples. Large collections of PCR primers thus have to be synthesized and individually applied to the miniscule reaction wells using robotized microdispensers. This is a sensitive and inflexible solution, since the arrays cannot be easily redesigned and each new application (biological species, type of assay etc) requires synthesis of a new large primer library. Due to the facts mentioned above, it is seen that a better method for manufacturing qPCR arrays is desired.

A successful technique for manufacturing DNA microarrays is the photolithographic method. The process begins by coating a flat substrate with a light-sensitive chemical compound that prevents coupling between the wafer and the first nucleotide of the DNA probe being created. The surface is then selectively exposed to light at specific locations. Subsequent flooding with a solution containing either adenine, thymine, cytosine, or guanine will cause coupling to occur only in those regions on the glass that have been deprotected through illumination. The coupled nucleotide also bears a light-sensitive protecting group, so that the cycle can be repeated. In this way, the microarray is built as the probes are synthesized through repeated cycles of deprotection and coupling until oligonucleotides of a desired length and sequence are obtained. This system produces very high-density microarrays and is described e.g. in U.S. Pat. No. 5,424,186 and U.S. Pat. No. 5,445,934. The method is well established and also described in numerous articles (Fodor S et al, Science. 251, 767-773 (1991), Jacobs, J. W. and Fodor, S. P., Trends Biotechnol. 12(1):19-26 (1994)). Photolithographic oligonucleotide synthesis traditionally uses lithographic masks to control the pattern of light projected onto the array. However, instruments referred to as maskless array synthesizers have been developed (U.S. Pat. No. 6,375,903). In these devices, the photolithographic masks are replaced by a computer controlled micromirror device of a type similar to that used in multimedia projectors. The light patterns projected onto the array surface are thereby completely controlled by software and the oligonucleotide sequences which are to be synthesized on the surface can be changed at any time with little effort.

An alternative method for in situ oligonucleotide synthesis is the inkjet method (see e.g., Blanchard, International Patent Publication WO 98/41531, published Sep. 24, 1998; Blanchard et al., 1996, Biosensors and Bioelectronics 11:687-690; Blanchard, 1998, in Synthetic DNA Arrays in Genetic Engineering, Vol. 20, J. K. Setlow, Ed., Plenum Press, New York at pages 111-123). This is also described in U.S. Pat. No. 7,013,221 B1. In this case, an ink jet printer head is used to apply nucleotides to specific locations on the array surface. This enables arbitrary sequences to be generated on the surface and provides a flexibility which is comparable to the maskless photolithographic method.

U.S. Patent Application No 2006/0147969 relates to the use of photolithographic preparation of high numbers of parallel sets of primers for PCR. Methods are provided for releasing oligonucleotides from the array surface. The only oligonucleotides that remain in position on the array are only those used for quality control. Unlike the invention described herein, the primers are not described to be prepared directly in reaction wells ready to use for PCR in situ.

U.S. Patent Application No 2005/0227263 describes a method for performing PCR amplification on a microarray. However, the patent describes a different approach based on a protocol which results in attachment of flanking universal primer sequences to a target DNA sequence. After completion of this step, the target sequence can be amplified using a pair of universal PCR primers. These primers are added to the general reaction mixture. The invention described herein significantly differs from this approach. No primers are added to the reaction mixture and there is no intermediate step where flanking universal primers are added to the target sequence. Instead, target specific primers are synthesized in each reaction well and released into solution using enzymatic degradation of a linker sequence.

It is therefore an object of the invention to provide an in situ oligonucleotide synthesis method for direct fabrication of polymerase chain reaction (PCR) primers in situ in PCR reaction wells. The present invention describes a solution where in situ oligonucleotide synthesis is employed for direct fabrication of PCR-primers in small volume PCR reaction wells on a microarray. Oligonucleotides, each containing an enzymatically degradable linker sequence and specific primer sequence, are generated on two or more spots in each well. Before execution of the PCR cycling program, the primer section of the oligonucleotides is released into solution using a heat incubation step.

Other objects and advantages will be more fully apparent from the following disclosure and appended claims.

### BRIEF SUMMARY OF THE INVENTION

The present invention can be briefly summarized as a method for parallel amplification and quantification of a plurality of target DNA sequences in a microarray format using the polymerase chain reaction (PCR). The method is based on the use of in situ oligonucleotide synthesis for direct fabrication of polymerase chain reaction (PCR) primers in situ in PCR reaction wells. Each oligonucleotide synthesized on the well surface contains an enzymatically degradable linker sequence and a specific primer sequence. During an initial heat incubation step, which is simply added to the normal PCR cycling program, the linker sequence is degraded and primers are released into solution. PCR is subsequently performed using standard reagents and cycling conditions. The invention may be used for manufacturing of quantitative PCR (qPCR) arrays containing a plurality of independent qPCR assays while eliminating the need for presynthesized primer libraries.

In a typical embodiment of the invention herein, a maskless photolithographic oligonucleotide synthesis apparatus is used to synthesize oligonucleotides on the bottom surfaces of small volume reaction wells. Oligonucleotides are synthesized on two spots in each well, one for the forward primer and one for the reverse primer. Each nucleotide is made to contain two parts. First a linker sequence containing U (uracil) bases is created. Synthesis continues with the addition of a specific primer sequence. The wells are loaded with a reaction mixture containing standard qPCR reagents (e.g. SYBR green, Taq polymerase, dNTPs, buffer) and DNA template. In addition, the enzyme uracil N-glycosylase (UNG) is added to the reaction mixture. Finally, the wells are sealed using a transparent plate. By adding a heat incubation step to the PCR cycling program (e.g. 50° C. for 10 min), primers will be released into solution before cycling begins.

Methods and materials for testing the present invention are provided herein. However, other related methods and materials, which are known in the art, can also be employed. Technical terms used herein have the same meaning as commonly accepted by those skilled in the art.

Other objects and advantages of the present invention will become obvious to the reader and it is intended that these objects and advantages are within the scope of the present invention.

### BRIEF DESCRIPTION OF THE DRAWINGS

FIG. 1 illustrates the general configuration of a qPCR microarray. Each well contains two or three oligonucleotide synthesis spots according to the invention herein.

FIGS. 2A and 2B are sectional side views of a reaction well during synthesis of linker sequences and specific primers sequences, respectively. The linker sequence may contain a restriction endonuclease recognition sequence, uracil bases cleavable by UNG (uracil N-glycosylase) or other cleavable moieties.

FIG. 3A illustrates sealing of the reaction wells using a transparent plate.

FIG. 3B illustrates the release of PCR primers using a heat incubation step before execution of the PCR cycling program.

### DETAILED DESCRIPTION OF THE INVENTION AND PREFERRED EMBODIMENTS THEREOF

The present invention generally relates to a method for detecting target DNA sequences using the polymerase chain reaction (PCR) in a microarray format. It is based on the use of in situ oligonucleotide synthesis for fabrication of PCR primer pairs directly in PCR reaction wells. This eliminates the need for presynthesized primer libraries and robotized dispension of primers in the reaction wells. Oligonucleotides, each containing a cleavable linker sequence and a specific primer sequence, are synthesized on two or more spots in each well. This leaves the oligonucleotides firmly attached to the bottom of the wells after synthesis. All wells are simultaneously loaded with a reaction master mix containing DNA template and standard reagents for quantitative PCR. The wells are then sealed and isolated from each other. During an initial heat incubation step, which is added to the normal cycling program, the linker is enzymatically degraded and PCR primers released into solution prior to execution of the PCR reaction.

In situ photolithographic oligonucleotide synthesis using a maskless array synthesizer is a well established method which is described in detail in several patents and articles (U.S. Pat. Nos. 5,445,934 and 6,375,903). Areas on a photosensitive surface are selectively exposed to light using a micromirror device. In areas exposed to light, photosensitive molecules are “unprotected” to enable binding of nucleosides containing photosensitive protective groups. By cycling through light exposure and binding steps, thousands of unique arbitrary sequences may be simultaneously synthesized at specific locations on the array surface. As a consequence of the synthesis method, the final oligonucleotides will be covalently attached to the surface.

Other in situ oligonucleotide synthesis methods may also be employed. In another embodiment of the invention, oligonucleotides are synthesized in situ using the ink jet method (Blanchard AP et al. Biosensors Bioelectron 11:687-690 (1996)). In this case, nucleoside triphosphates are selectively dispensed at specific locations using an ink jet printer. This will also leave the finished oligonucleotides covalently attached to the substrate.

The term “in situ oligonucleotide synthesis” used herein refers generally to methods which synthesize oligonucleotide sequences directly on e.g. a microarray surface. This is opposed to methods which employ a pre-synthesized library of oligonucleotides which is later mechanically dispensed or spotted to specific locations.

The term “PCR” as used herein refers to the polymerase chain reaction, which is a standard method in molecular biology. It is used to amplify (copy) a selected region of a DNA molecule. In its basic embodiment, the method uses a pair of oligonucleotides, “primers”, which specifically bind (anneal) to specific locations of a longer DNA molecule. The region in between and including the primers is copied several thousand fold using a cyclic enzymatic reaction based on the use of a heat-tolerant DNA polymerase such as Taq polymerase.

The terms “quantitative PCR” or “real-time PCR” as used herein refers to methods where the PCR reaction is combined with fluorescence chemistry such as SYBR green or Taqman, to enable real-time monitoring of the amplification reaction using detection of a fluorescent light signal. During execution of the PCR cycling program, the samples are excited using a light source. A fluorescent signal, indicating the amount of PCR amplification product produced, is monitored in each reaction well using a photodetector or CCD/CMOS camera. As opposed to normal PCR, this principally simple extension of the basic method allows precise quantification of specific DNA sequences in a sample.

The term “template DNA” used herein refers to any DNA molecule which can be amplified using the PCR reaction. This includes, but is not limited to, genomic DNA and complementary DNA (cDNA) produced from RNA using reverse transcription. The method described herein may thus be used e.g. for parallel genotyping of single nucleotide polymorphisms (SNPs) or quantification of messenger RNA (mRNA) expression in any organism.

The term “primer” or “primer pair” as used herein refers to short oligonucleotides (typically 18-25 bp) which are used in PCR to select which DNA sequence should be amplified. The size of typical oligonucleotides is well within the boundaries of maskless photolithographic oligonucleotide synthesis, which can be used to synthesize oligonucleotides of 50 bp length or more.

As to a further discussion of the manner of usage and operation of the present invention, the same should be apparent from the above description. Accordingly, no further discussion relating to the manner of usage and operation will be provided.

With respect to the above description then, it is to be realized that the optimum dimensional relationships for the parts of the invention, to include variations in size, materials, shape, form, function and manner of operation, assembly and use, are deemed readily apparent and obvious to one skilled in the art.

Therefore, the foregoing is considered as illustrative only of the principles of the invention. Further, since numerous modifications and changes will readily occur to those skilled in the art, it is not desired to limit the invention to the exact construction and operation shown and described, and accordingly, all suitable modifications and equivalents may be resorted to, falling within the scope of the invention.

### Example 1

### Simultaneous Synthesis of Unique Oligonucleotide Sequences Containing UNG-Degradable Linkers at Specific Locations on the Array

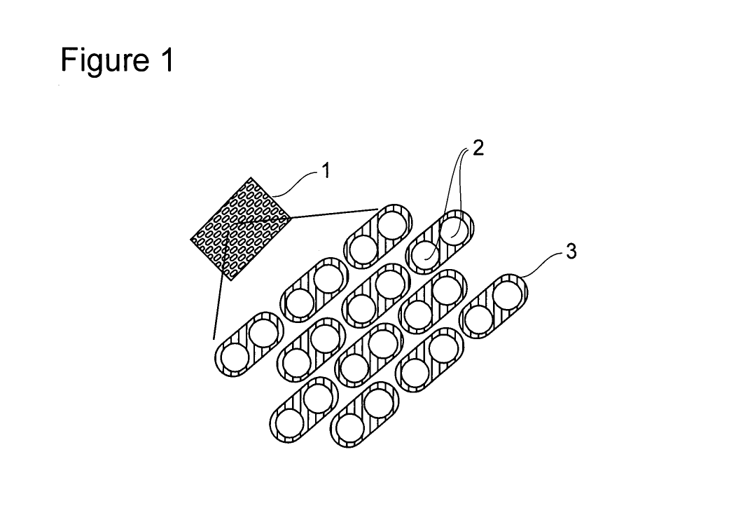

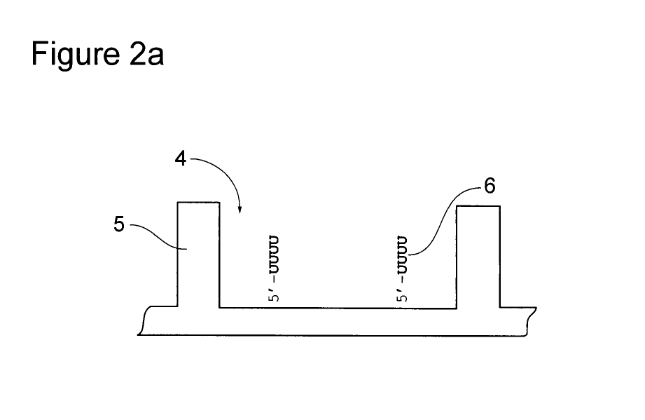

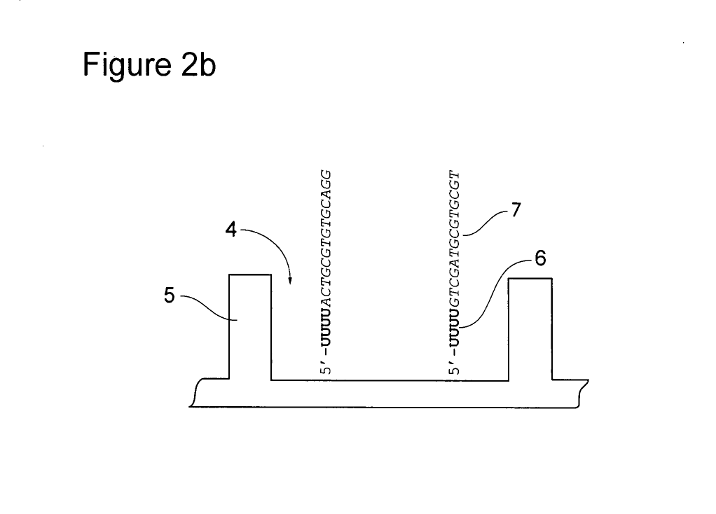

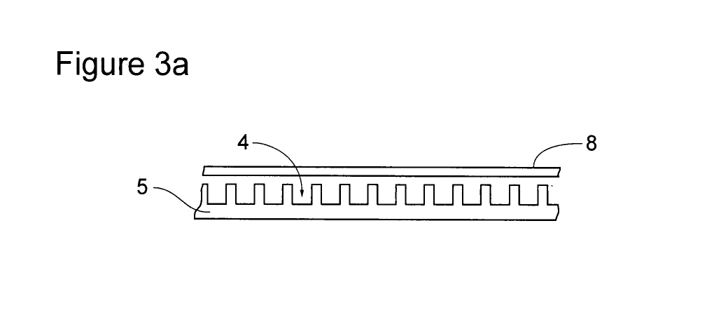

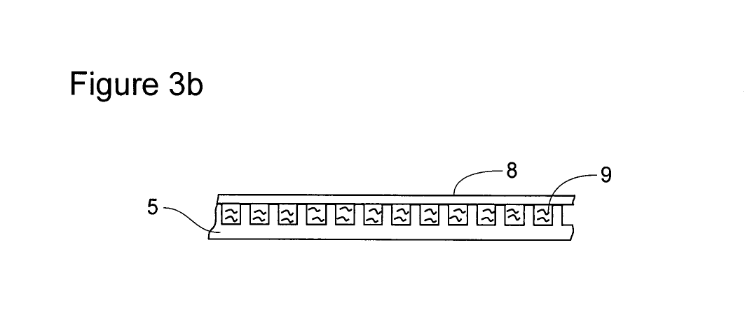

In one preferred embodiment, the bottom surfaces of small open wells on a microarray are prepared with surface chemistry (linker molecules) which enables in situ oligonucleotide synthesis using a maskless photolithographic array synthesizer. FIG. 1 is a schematic view of the general arrangement of such a device. The array 1 contains wells in a tightly packed pattern. Oligonucleotides are synthesized on two spots 2 (one for each primer) in each well 3 (shown in a magnified view in FIG. 1). Oligonucleotides are synthesized in the 5′-3′ direction, leaving a free 3′ end, however 3′-5′ synthesis is also conceivable as both methods are well established. First, identical linker sequences containing U (uracil) bases are synthesized in each spot. The linker sequences enable later release of the primers using enzymatic degradation. FIG. 2A is a side view of a single well 4 on an array 5 during synthesis of the linker sequences 6. Second, shown in FIG. 2B, the actual primers 7, which will be unique in each location, are synthesized. The primer precursor oligonucleotides are by now firmly attached to the surface and the open wells can be loaded with standard qPCR reagents (e.g. dNTPs, hotstart taq polymerase, SYBR green, buffer) and template in a single pipetting step without risk of primer contamination between wells. In addition, the enzyme uracil N-glycosylase (UNG) is added to the reaction mixture. The wells are now sealed using a transparent glass plate 8 or similar as previously described (see e.g. Dahl A et al, Biomed Microdevices. 2007 June; 9(3):307-14) (FIG. 3a). qPCR can now be performed as usual with the addition of an initial 50° C. incubation step which activates the UNG enzyme. This will result in degradation of the linker sequences, causing primers 9 to be released into solution before execution of the PCR reaction (FIG. 4B). A standard real-time qPCR reaction is now performed; the samples are excited using a light source, and the amount of PCR product produced is continuously determined by monitoring the fluorescent signal in the wells.

### Example 2

### Using a Linker Containing a Restriction Endonuclease Recognition Sequence

In other embodiments, other linkers may be employed. Using a linker sequence containing a restriction endonuclease recognition sequence, primers may be released using restriction cleavage. In this case, complementary oligonucleotides are annealed to the restriction linker sequences after synthesis. Oligonucleotide synthesis in the 3′-5′ direction may be more appropriate in this case, although both directions are conceivable. Before loading and sealing of the array, a restriction enzyme is added to the reaction mixture. The primers are released using an initial heat incubation step, normally at 37° C., before execution of the PCR program. Several enzymes can be considered. An example of a suitable enzyme is BamHI, which has good activity in PCR buffer. Assuming synthesis in the 3′-5′ direction this leaves only a single G base in the 3′ end of the primers after cleavage. Consequently, primers will have to be designed to carry a G base in the 3′ end. Since G bases occur fairly frequently even in non-GC-rich genomic regions, this is not a limiting factor for most applications.

### Example 3

### Using Probe-Based Fluorescent Detection

In yet another embodiment, oligonucleotides are synthesized on three different locations/spots in each reaction well. In this case, a fluorescent oligonucleotide probe such as a Taqman probe, is synthesized in each well in addition to the two PCR primers. The probe is synthesized according to the same principle as the primers and the same type of linker sequence (e.g. uracil containing) will be used. Also in this case, standard qPCR reagents can be used, and the remaining procedures will essentially be identical to what has been described in EXAMPLE 1 or 2.

What is claimed is:

1. A method for detecting target DNA sequences in a sample using the polymerase chain reaction independently in a plurality of wells, comprising:
a) synthesizing oligonucleotides in situ in at least two spots in each well of the plurality of wells, each of the oligonucleotides comprising a cleavable linker sequence and a specific primer sequence in a solution;
b) simultaneously loading the plurality of wells with a reaction master mix containing DNA template and polymerase chain reaction reagents;
c) sealing the wells and isolating the wells from each other;
d) heating the wells in an initial heat incubation step so that the cleavable linker sequences are enzymatically degraded and the primer sequences are released into solution; and
e) utilizing the polymerase chain reaction to detect whether the target DNA sequences are present.

2. The method for detecting target DNA sequences according to claim 1, wherein the bottom surfaces of the wells are prepared with surface chemistry enabling in situ oligonucleotide synthesis using a maskless array synthesizer.

3. The method for detecting target DNA sequences according to claim 2, wherein the samples are excited with a light source, and the amount of polymerase chain reaction product in each well is determined by monitoring fluorescent signal in the wells.

4. The method for detecting target DNA sequences according to claim 1, wherein the cleavable linker sequences are degradable by uracil N-glycosylase.

5. The method for detecting target DNA sequences according to claim 1, wherein the cleavable linker sequences contain a restriction endonuclease recognition sequence and primers are released during restriction cleavage.

6. The method for detecting target DNA sequences according to claim 1, wherein oligonucleotides are synthesized at three spots in each well, wherein one spot is for a forward primer, one spot is for a reverse primer, and one spot is for a fluorescent probe.

7. The method for detecting target DNA sequences according to claim 6, wherein the fluorescent probe is a Taqman probe.

8. The method for detecting target DNA sequences according to claim 1, wherein oligonucleotides are synthesized on two spots in each well, one spot for a forward primer and one spot for a reverse primer.

9. The method for detecting target DNA sequences according to claim 1, wherein the wells are sealed using a transparent plate.

10. A method of preparing a quantitative polymerase chain reaction array, comprising:
a) synthesizing oligonucleotides in at least two spots in each well of the plurality of wells, each of the oligonucleotides comprising a cleavable linker sequence and a specific primer sequence;
b) simultaneously loading the plurality of wells with a reaction master mix containing DNA template and polymerase chain reaction reagents;
c) sealing the wells and isolating the wells from each other;
d) heating the wells in an initial heat incubation step so that the cleavable linker sequences are enzymatically degraded and the primer sequences are released into solution; and
e) utilizing the polymerase chain reaction to amplify the oligonucleotides to detect whether target DNA sequences are present.
